(19) United States
(12) Patent Application Publication
(10) Pub. No.: US 2024/0280531 A1
(43) Pub. Date: Aug. 22, 2024

(54) Sensor having contoured membrane

(71) Applicants: Sahan RANAMUKHAARACHCHI, Vancouver (CA); Victor Javier CADARSO BUSTO, Mentone, Victoria (AU); Esma DERVISEVIC, Clayton, Melbourne (AU); Allison OLENGINSKI, Silverthorne, CO (US); Proton Intelligence Inc., Gibsons (CA)

(72) Inventors: Sahan Ranamukhaarachchi, Vancouver (CA); Victor Javier Cadarso Busto, Mentone (AU); Esma Dervisevic, Clayton (AU); Allison Olenginski, Silverthorne, CO (US)

(73) Assignee: Proton Intelligence Inc., Gibsons, BC (CA)

(21) Appl. No.: 18/571,809

(22) PCT Filed: Jul. 14, 2022
(86) PCT No.: PCT/US2022/037198

§ 371 Date: Dec. 19, 2023

Related U.S. Application Data
(60) Provisional application No. 63/222,936, filed on Jul. 16, 2021.

(51) Int. Cl. G01N 27/333

(52) U.S. Cl. CPC G01N 27/333

(57) ABSTRACT

A sensor is provided and includes a substrate, a patterned electrode, and a membrane material. The patterned electrode is deposited on the substrate and has a conductive surface. The membrane material is deposited on the conductive surface of the patterned electrode and not on the planar substrate. The membrane material forms a three-dimensional membrane layer that covers the conductive surface of the patterned electrode.

### CROSS-REFERENCE TO RELATED APPLICATIONS

This application is related and claims priority to U.S. Prov. Pat. App. Ser. No. 63/222,936 filed on Jul. 16, 2021, which is incorporated herein by reference for all purposes.

### BACKGROUND OF THE INVENTION

It is invaluable to be able to obtain real-time, continuous sensing data in vivo. Continuous sensing data from a patient allows a healthcare provider to tailor therapy to the patient, and facilitates early administration of interventional measures.

Patients with acute heart failure (HF), for example, are often prescribed diuretic therapy, renin-angiotensin-aldosterone system (RAAS) blockers, angiotensin II receptor blockers (ARBs), angiotensin-converting enzyme inhibitors (ACEi), and the like These therapies influence how the body handles potassium. If the dose prescribed is too high, the patient may be at risk of developing hyperkalemia—particularly if the patient has comorbidities such as chronic renal insufficiency. Currently, the only way to monitor a patient's potassium levels—on an inpatient or outpatient basis—is with laboratory testing using arterial blood, which is time consuming, expensive and must be conducted on a regular (and frequent) basis. This does not encourage or facilitate patient compliance with monitoring and, thus, is not reliably used in outpatient care. Health care providers, therefore, often err on the side of caution and prescribe medication at a dose that creates less risk of hyperkalemia—but may not have the desired or full therapeutic effect. Thus, the patient's quality of life might not be significantly improved, despite adherence to a prescribed protocol. However, if the patient's potassium levels could be monitored in real-time, the dosing regime could be optimized for the specific patient, improving patient outcome. And, in the event that a continuously monitored patient develops even mild hyperkalemia, swift intervention can minimize negative outcomes.

In some circumstances, the position of a sensor in a patient's body can allow health care providers to intervene before a patient experiences symptoms associated with his or her illness. Surgery site infections, for example, are experienced by as many as 5% of surgical patients. Some surgeries, particularly those involving the intestines, result in deep incisional surgical site infection of 25% or more of patients. However, if one or more sensors placed at, or near the surgical site were able to monitor for signs of infection (inflammation, pH change, temperature change, and the like), treatment (antibiotics, wound cleaning, etc.) could begin before a patient experiences noticeable signs of infection, and without having to undress/redress the incision site multiple times each day.

While numerous in vivo sensors currently exist for a wide variety of biomarkers, most in vivo sensors are miniaturized versions of ex vivo sensors that are modified to be biocompatible. Typically, electrochemical sensors designed for use in vivo are flat or planar structures, or wire-based sensors (as often found in continuous glucose monitoring (CGM) sensors).

Electrochemical sensors generally include one or more “sensing” or working electrodes and may also include one or multiple reference electrodes. In some electrochemical sensors, a counter electrode may be present. Many sensors include a membrane—whether that membrane is an integral part of the sensor and, for example, selective for a biomarker; or whether that membrane is primarily intended to render the sensor biocompatible. When these sensors are placed in a sample, the ion/molecule to be detected (the analyte) has to be transported across the membrane before any reaction can occur at the sensing or working electrode(s).

Whilst conventional miniaturization does provide several benefits such as improving sensor portability, analyte flux across the sensor surface, multiplexing capability, and ability to analyze samples in microliter (or even less) volume, the miniaturization often is accompanied by challenges impacting the sensor performance. These challenges mainly originate from decreased surface area that interacts with the sample fluid, particularly when the sensor is configured for analyte detection, and can result in higher background noise, poorer sensor lifetime, detection limit, sensitivity, dynamic range, and response reproducibility. In an event of biofouling, this miniature sensing area available for analyte detection is downsized further. Additionally, in vivo sensors often cause an inflammatory response in surrounding tissue, particularly when the sensor(s) remains in place for continuous monitoring.

Thus, there remains a need for miniaturized sensors capable of quantifying/monitoring without compromising the required analytical performance characteristics including sensitivity, dynamic range, accuracy of measurement, stability, detection limit of miniaturized sensors—whether ex vivo, in vivo, in vitro, or in other uses such as in environmental monitoring. Moreover, there is a particular need for miniaturized, implantable sensors capable of continuous monitoring of one or more conditions, analytes, or biomarkers in vivo.

### SUMMARY OF THE INVENTION

The present invention provides solutions to the above-described problems associated with the art. In a first embodiment, the present invention provides a sensor that includes a substrate (for example a patterned substrate), a patterned electrode (for example a working electrode), and a membrane material (for example an analyte-selective membrane material). The patterned electrode is deposited on the substrate and has a conductive surface. The membrane material is preferably biocompatible and is deposited on the conductive surface of the patterned electrode and not on the planar substrate. The membrane material forms a three-dimensional membrane layer that covers the conductive surface of the patterned electrode.

In a second embodiment, the present invention provides another sensor (optionally including components of the first embodiment) including: a substrate; a first patterned, three-dimensional working electrode deposited on the substrate (e.g. the three-dimensional working electrode having a plurality of surfaces, wherein said plurality of surfaces are not in contact with the substrate); a reference electrode deposited on the substrate; and a first membrane layer, wherein said membrane layer is deposited on the plurality of surfaces of the first three-dimensional working electrode.

In a third embodiment, the present invention provides a dual-sided sensor (optionally including components of the first and/or second embodiments) including: a substrate having a first side and a second side; a first patterned, three-dimensional working electrode deposited on the first side of the substrate, the three-dimensional working electrode having a plurality of surfaces, wherein said plurality of surfaces are not in contact with the substrate; a first membrane layer, said first membrane layer being deposited on the plurality of surfaces of the first three-dimensional working electrode; a second patterned, three-dimensional working electrode deposited on the second side of the substrate, the second three-dimensional working electrode having a second plurality of surfaces, wherein said second plurality of surfaces are not in contact with the substrate; a second membrane layer, said second membrane layer being deposited on the plurality of surfaces of the second three-dimensional working electrode; and a reference electrode, said reference electrode not being in contact with the first or second membrane layer.

In an additional embodiment, the present invention provides a method of causing two-dimensional or three-dimensional transport (or increasing transport) of an ion through a membrane to a sensor surface, the method comprising the steps of: providing a sensor as described in any sensor or membrane described herein; and exposing the membrane to a solution comprising the ion.

In a further embodiment, a method of determination an ion in a solution comprising the ion is provided. The method comprising the steps of: providing a sensor as described herein; exposing the membrane to the solution comprising the ion; and electrochemically determining a property of the ion. Thereby determining an ion in the solution.

### BRIEF DESCRIPTION OF THE DRAWING

FIG. 1 shows a side view of a sensor according to the art.

FIG. 2 shows a top and two end views of sensors according to the present invention.

FIG. 3 shows a top view and side view of sensors according to the present invention.

FIG. 4 shows a top view and side view of sensors according to the present invention.

FIG. 5 shows a top view, bottom view, and side view of sensors according to the present invention.

FIG. 6 shows a top view, bottom view, and side view of sensors according to the present invention.

FIG. 7 shows a top view and side view of sensors according to the present invention.

FIG. 8 shows side views of various sensors according to the present invention.

FIG. 9 shows side views of various sensors according to the present invention.

### DETAILED DESCRIPTION OF THE INVENTION

Aspects of the present invention are described herein with respect to a potentiometric sensor and, more specifically, an ion-selective potentiometric sensor. However, it should be appreciated that the embodiments described herein are non-limiting and that this invention could be implemented in any electrochemical sensor (including but not limited to potentiometric sensors, conductometric sensors, amperometric/voltammetric sensors, ISFETs, affinity-based sensors, and the like). The membranes described could be also implemented in physical sensors (including but not limited to temperature sensors, motion sensors, strain sensors, pressure sensors, telemetry units, and the like) and electrical sensors (including but not limited to impedance sensors, strain sensors, pressure sensors, neural interfaces, and the like), or any other sensor that includes a working electrode covered by a membrane, without departing from the scope of the invention. Generally, operation of the sensor requires transport of an ion/molecule/substance across a membrane so that a reaction may occur, resulting in detection and/or monitoring of the desired reaction by any chemical, physical, or electrical methods/techniques.

As used herein, “sensor” refers to a device, module, machine, or subsystem that detects changes or events in its environment. While the embodiments disclosed herein are generally described with respect to specific implementations of electrochemical sensors, it should be appreciated that any type of physical, electrical, or electrochemical sensor that utilizes a membrane that requires some material (ions, molecules, etc.) that is present in the sample solution to cross the membrane in order for sensing or a reaction to occur. The sensor may be configured for any type of sensing including, but not limited to, an environmental sensor, a physical sensor, an implantable sensor, a bio-sensor (having a biorecognition element), laboratory equipment, or the like.

“Substrate” as used herein is the material on which one or more electrodes are fabricated.

“Transducer” generally refers a device that converts energy from one form to another. As used herein, the term refers to one or more electrodes contained in a sensor. In the context of an ion-sensing potentiometric sensor, the transducer includes a working electrode and a reference electrode.

“Analyte” as used herein refers to any substance, ion, or material, present in a sample fluid, that is measured by the sensor(s).

As used herein, the phrase “membrane material” refers to analyte-selective membranes, bio-selective membranes, biocompatible membranes, anti-fouling layers/coatings, drug-eluting materials used for coating, and the like. An analyte selective membrane is a membrane that allows the desired analyte to pass across the membrane, but does not allow other materials to pass across the membrane. Bio-selective membranes include, but are not limited to, any membrane that is selective for a target analyte such as an ion or other molecules Exemplary bio-selective membranes include ion-selective membranes, gas-selective membranes, and bio-molecule selective membranes. Preferably, membrane material for use in an in vivo sensor does not elicit an adverse reaction from a patient's body, and is resistant to degradation for at least the expected/desired life of the sensor. When applied to an electrode, membrane material has two sides: a first side that is in contact with a sample, and a second side that is in direct contact with conductive electrode material (for the purposes of a solid-state ion selective electrodes or in the case of other ion-selective electrodes the membrane material can be in contact with an inner solution etc.).

The phrase “conductive material,” as used herein, refers to electrically conductive material deposited on the surface of a transducer or substrate to form an electrode. Exemplary conductive materials include platinum, gold, palladium, rhodium, rhenium, ruthenium, osmium, iridium, platinated platinum, precious metal alloys, graphite, carbon, titanium, brass, conductive polymers, or the like It should be appreciated that one or more conductive materials may be used in the formation of electrodes for use in each sensor. For example, the working electrode and reference electrode in a sensor may be comprised of different conductive materials. In some embodiments, multiple conductive materials may be deposited as part of a single electrode.

“Sample,” as used herein, may be any fluid that is under test or monitoring by the sensor. In the case of an implantable, transcutaneous biosensor, the sample may be interstitial fluid. In the case of other implantable sensors, the sample may be blood, urine, gas, or other bodily fluids. In the case of environmental sensors, for example, the fluid may be a liquid solution or a gas.

“Topography,” as used herein, refers to the formation or arrangement of two and/or three-dimensional surface features that project from the substrate of the sensor. Every feature, such as an electrode formed of conductive material, added to or deposited on the surface of a substrate or on the contours of a substrate has a topography.

It should be appreciated that the inventions described herein may be manufactured or created using any known substrate and/or transducer material. For implantable or wearable sensor applications, it should be appreciated that biocompatible materials should be selected. However, the embodiments described herein are applicable to any sensor that includes a patterned electrode covered by a membrane material, without departing from the scope of the invention.

Any known technique for forming electrodes on a substrate may be used including, but not limited to, lithographic techniques, ink-jet printing, screen-printing, three-dimensional printing, lift-off, evaporation, sputtering, electroplating or the like. It should be appreciated that, in some embodiments, the electrode(s) may be formed on a sacrificial layer which is later removed. Finally, the membrane material layer disclosed may be formed or molded independently and affixed to the surface of the electrode, may be created using lithographic techniques, two dimensional and three-dimensional printing techniques, dip coating, spin coating, spray coating, or the like. The specific methods and techniques used to create the sensor may be varied without departing from the scope of the invention.

FIG. 1 (prior art) illustrates a conventional solid state ion-selective electrode (ISE) 101 having a transducer that includes a working electrode 103 made of a conductive material, such as carbon, that is deposited on a substrate 105. In a potentiometric sensor, this solid-state ISE would be paired with a reference electrode to measure potential difference between the two electrodes. In some embodiments of an amperometric sensor, the transducer would include a solid-state ISE, a working electrode, and a counter electrode. Much of the surface area of the conductive layer is covered by an insulator, leaving only a small area of the conductive layer 103 exposed for interacting with the membrane 107. The exposed area of the conductive layer and the insulator are covered by an ISE membrane 107. The ISE membrane is selective for a specific ion, for example Ca⁺. The ISE membrane 107 has a first side that contacts the sample, and a second side that is in contact with both the insulator 109 and the conductive layer 103 exposed through the insulator. While the surface area of the bio-selective membrane 107 that contacts the sample on the first side is significant, relatively little of the second surface of the ISE membrane contacts the conductive layer 103 or conductive coating thereof (e.g. a Ag/AgCl coating). Thus, the amount of the target ion transferred through the selective membrane 107 may not generate a significant signal change, even when the sample fluid contains an abundance of the target ion.

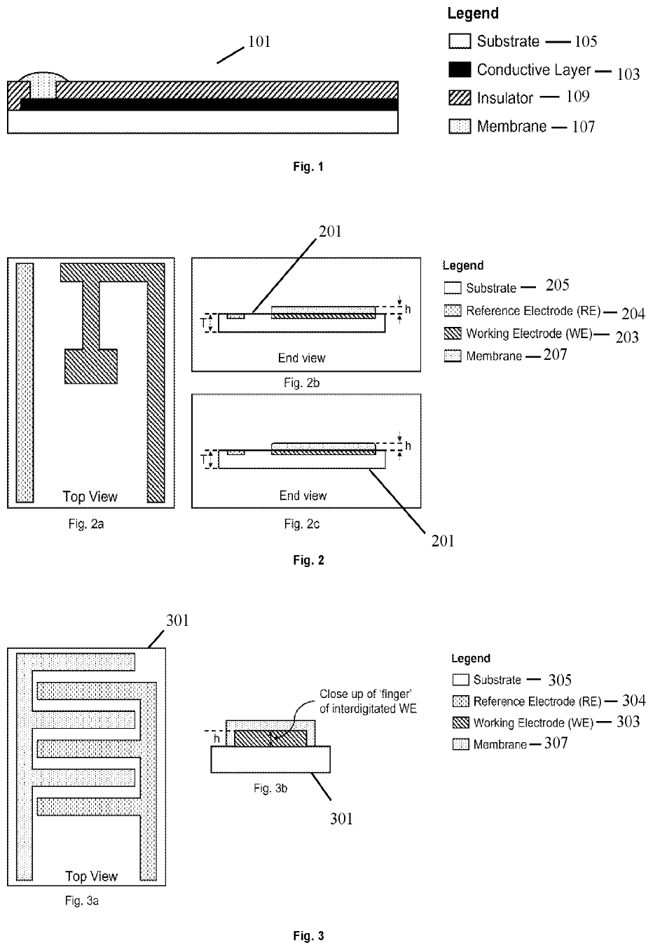

FIGS. 2a, 2b, and 2c illustrate planar sensors 201 having a planar patterned electrode 203 in accordance with the present invention. In FIG. 2a, a top view of the planar sensor is shown. The biocompatible substrate 205 having a thickness T was patterned using a conventional lithographic technique, and conductive material 203 was subsequently deposited using conventional deposition techniques to form a transducer having a planar surface. The depth of the substrate 205 patterning/conductive material deposition is shown in FIGS. 2b and 2c. The conductive material 203 does not extend beyond the lateral dimensions of the substrate 205 on which the conductive material 203 is deposited.

As illustrated in FIGS. 2a, 2b, and 2c, according to an embodiment of the present invention, a membrane material 207, such as a bio-selective membrane or biocompatible membrane is deposited on, or covers the surface of the patterned working electrode 203, but does not contact the reference electrode 204 or the substrate 205. The membrane material 207 may be selective for a material including, but not limited to, ions, biomarkers, target molecules, or the like. In some embodiments, the membrane material 207 may be biocompatible and not selective for a material. As illustrated in the cross-sections of FIGS. 2b and 2c, the membrane material 207 has a height h (shown), a first side having a plurality of surfaces that are in contact with a sample fluid, and a second side that is in direct contact with the conductive surface of the planar working electrode 203. Thus, the membrane material 207 creates a three-dimensional structure on the otherwise planar or flat sensor, and maximizes the ratio of membrane surface area to membrane/working electrode contact area. It is preferable that the height or thickness of the membrane material 207 be optimized for the specific membrane material used in the sensor and the specific application of the sensor 201.

In FIGS. 2a, 2b, and 2c, the overall surface area of the membrane material 207 in contact with the sample is significantly increased relative to the solid-state ion-selective electrode structure shown in FIG. 1 (prior art), thus decreasing sensor response time by at least 10%, 20%, 30%, 40%, 50% or more. Sensor response time is decreased because transport time across the membrane is decreased as a result of 1) a less thick (or thinner) membrane layer, 2) more surface area of the membrane being in contact with the sample solution, and 3) more surface area of the membrane being in contact with the surface of the electrode. Stated another way, more of the target substance transported across the membrane makes contact with the working electrode, in comparison to conventional sensor structure illustrated in FIG. 1, in which a relatively small percentage of the target substance transported across the membrane actually makes contact with the surface of the working electrode. Additionally, because the membrane layer is thinner relative to conventional electrode designs, and more of the membrane layer is in contact with the sample, “warm-up” time for the sensor is also reduced by at least 1%, 2%, 3%, 4%, 5%, 6%, 7%, 8%, 9%, 10%, 15%, 20%, 25%, 30%, 40%, 50% or more because more of the target molecule will be transported across the membrane in shorter time. This means that the sensor will be ready to use and provide consistent, accurate, and precise results more quickly than conventional designs, which utilize relatively thick layers of membrane, and have relatively small areas of the electrode layer in contact with the membrane for material transfer. Further, it should be appreciated that the texture of the membrane surface may be modified using known techniques to improve or optimize wetting properties of the membrane.

FIG. 2b illustrates an embodiment of the present invention in which the membrane 207 has multiple edges and sharp vertices. FIG. 2c illustrates an embodiment in which the edges of the membrane 207 are contoured or rounded to reduce the effects of biofouling. It should also be appreciated that, in some embodiments, the surface of the membrane 207 may be further micro- or nano-patterned using known micro-/nano-patterning techniques to increase the effective surface area of the membrane 207 material in contact with sample. Further, it should be appreciated that the exact contours of the membrane 207 may be varied over the surface of the patterned working electrode to ease sensor 201 insertion/removal if the sensor is designed to be implanted or to be subcutaneous, to enhance the structural rigidity of the overall sensor structure without adding bulk or mass, or to increase the surface area of the membrane 207 in contact with the sample. It should also be appreciated that the thickness of the membrane 207 may be consistent in some applications, and varied in other applications, without departing from the scope of the invention.

FIG. 3a illustrates an embodiment of the present invention in which the sensor 301 includes interdigitated working 303 and reference 304 electrodes that have been printed using, for example, inkjet printing or screen-printing techniques and suitable conductive materials on a planar, biocompatible substrate 305. The membrane material 307 is applied to the surfaces of the working electrode 303, does not cover the reference electrode 304, and only contacts the area of the substrate 305 immediately adjacent to the working electrode 303.

Interdigitated electrodes 303, 304 facilitate miniaturization because they dramatically increase the surface area of electrode available for sensing over conventional patterned electrodes deposited on the same sized substrate.

A cross section of one “finger” of the interdigitated working electrode 303 is illustrated in FIG. 3b. As shown in FIG. 3b, while the substrate 305 is planar, each of the interdigitated electrodes 303. 304 projects perpendicularly from the substrate 305, and has a height or thickness. Thus, the interdigitated electrodes 303, 304 are three-dimensional, and have a plurality of surfaces that are not in contact with the substrate 305. In cross-section, each finger appears rectangular. However, it should be appreciated that the complex geometry of interdigitated electrodes may vary without departing from the scope of the invention. The membrane material 307 is applied to all exposed surfaces of the working electrode 303 using the techniques described herein. Thus, the membrane material 307 follows the contours of the three-dimensional working electrode 303, and is also three-dimensional, allowing multiple angles for transport of target ions/molecules across the membrane 307. The height or thickness of the membrane is preferably optimized for the target ion/molecule and sensor application. In some embodiments, the height/thickness of the membrane material 307 is consistent on all working electrode 303 surfaces. In other embodiments, the membrane material 307 may be applied in different thicknesses/heights on different surfaces or aspects of the working electrode 303 material. In still other embodiment, the thickness/height of the membrane material 307 may have a variable profile along the surface of the electrode material, increasing the surface area of the membrane in contact with sample.

In FIGS. 3a and 3b the overall surface area of the membrane material 307 in contact with the sample is significantly increased relative to the solid-state ion-selective electrode 303 structure shown in FIG. 1 (prior art), thus decreasing sensor response time by at least 1%, 2%, 3%, 4%, 5%, 6%, 7%, 8%, 9%, 10%, 15%, 20%, 25%, 30%, 40%, 50% or more. Sensor response time is decreased because transport time across the membrane 307 is decreased as a result of 1) a less thick (or thinner) membrane layer 307, 2) more surface area of the membrane 307 being in contact with the sample solution, and 3) more surface area of the membrane 307 being in contact with the surface of the electrode 303. Stated another way, more of the target substance transported across the membrane 307 makes contact with the working electrode 303, in comparison to conventional sensor structure illustrated in FIG. 1, in which a relatively small percentage of the target substance transported across the membrane 307 actually makes contact with the surface of the working electrode 303. Additionally, because the membrane layer 307 is thinner relative to conventional electrode designs, and more of the membrane layer 307 is in contact with the sample, “warm-up” time for the sensor is also reduced by at least 1%, 2%, 3%, 4%, 5%, 6%, 7%, 8%, 9%, 10%, 15%, 20%, 25%, 30%, 40%, 50% or more because more of the target molecule will be transported across the membrane more quickly. This means that the sensor will be ready to use and provide consistent, accurate, and precise results more quickly than conventional designs, which utilize relatively thick layers of membrane, and have relatively small areas of the electrode layer in contact with the membrane for material transfer. Further, it should be appreciated that the texture of the membrane surface 307 may be modified using known techniques to improve or optimize wetting properties of the membrane 307.

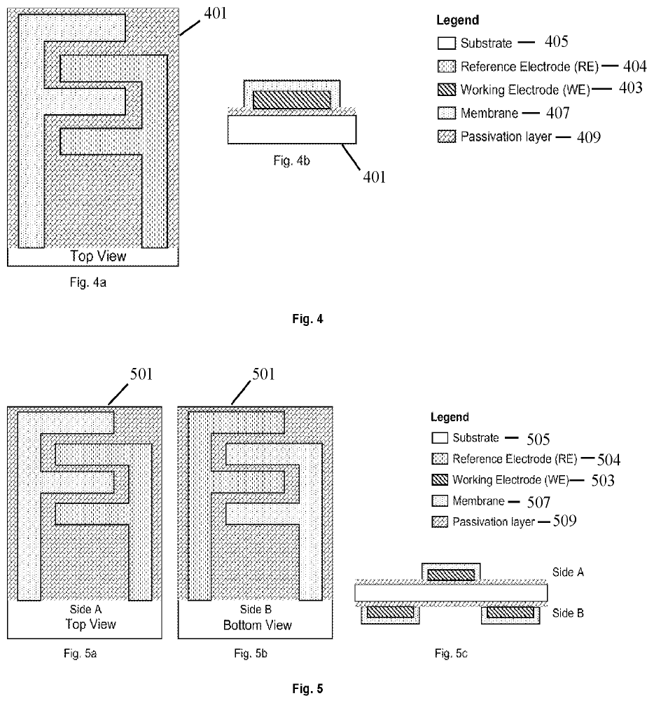

FIGS. 4a and 4b illustrate an exemplary interdigitated sensor 401 in accordance with the present invention. As illustrated in FIG. 4a, the sensor 401 includes a working electrode 403 and a reference electrode 404. The working electrode 403 and reference electrode 404 are electrically separated from one another by a passivation layer 409 on the top surface of the substrate 405. The passivation layer 405 serves as an insulating layer, and allows for interaction between the selected target ion/molecule and the bio-selective membrane 407. The passivation layer 407 also helps to avoid false negative signals and interference from the substrate. In some implementations, the passivation layer 409 may be SiO2, Si3N4, SU-8, polyamide, parylene, or any other non-conductive (or insulating) material appropriate for the application, applied via conventional deposition techniques in a thickness optimized for the sensor application. While a passivation layer 409 is illustrated in FIGS. 4A and 4B, it should be appreciated that, in some embodiments, the substrate 405 may serve as a passivation layer 409. In other embodiments, the passivation layer 409 is optional or absent.

As illustrated in FIG. 4b, which shows the cross-section of a single “finger” of the interdigitated working electrode 403, the conductive material of the working electrode 403 can be deposited on the surface of the substrate 405, and has a height or thickness. The working electrode 403 can be isolated from the reference electrode 404 by inclusion of a passivation layer 409 disposed between the respective electrodes and the substrate 405. In the case of a conductive substrate 405, a passivation layer 409 could be present between either or both the electrodes 403, 404 and the substrate 405. Preferably, the passivation layer 409 has a height/thickness less than that of the working electrode 403. The membrane material 407 is deposited on the planar surfaces of the working electrode 403 exposed to the sample in a thickness or a variable thickness optimized for the membrane 407 and desired sensor characteristics, thus forming a membrane layer 407 that matches the contour or topography of the working electrode 403.

Because the membrane material 407 follows the contours of the three-dimensional working electrode 403, it is also three-dimensional, allowing multiple angles for transport of target ions/molecules across the membrane and increased surface area. The height or thickness of the membrane 407 is preferably optimized for the target ion/molecule and sensor application. In some embodiments, the height/thickness of the membrane material 407 is consistent on all working electrode surfaces 403. In other embodiments, the membrane material 407 may be applied in different thicknesses/heights on different surfaces or aspects of the working electrode material 403. In still other embodiment, the thickness/height of the membrane material 407 may have a variable profile along the surface of the electrode material 403, increasing the surface area of the membrane 407 in contact with the sample.

In FIGS. 4a and 4b, the overall surface area of the membrane material 407 in contact with the sample is significantly increased relative to the solid-state ion-selective electrode structure shown in FIG. 1 (prior art), thus decreasing sensor response time by at least 1%, 2%, 3%, 4%, 5%, 6%, 7%, 8%, 9%, 10%, 15%, 20%, 25%, 30%, 40%, 50% or more. Sensor response time is decreased because transport time across the membrane is decreased as a result of 1) a less thick (or thinner) membrane layer, 2) more surface area of the membrane being in contact with the sample solution, and 3) more surface area of the membrane being in contact with the surface of the electrode. Stated another way, more of the target substance transported across the membrane makes contact with the working electrode, in comparison to conventional sensor structure illustrated in FIG. 1, in which a relatively small percentage of the target substance transported across the membrane actually makes contact with the surface of the working electrode. Additionally, because the membrane layer is thinner relative to conventional electrode designs, and more of the membrane layer is in contact with the sample, “warm-up” time for the sensor is also reduced by at least 1%, 2%, 3%, 4%, 5%, 6%, 7%, 8%, 9%, 10%, 15%, 20%, 25%, 30%, 40%, 50% or more because more of the target molecule will be transported across the membrane more quickly. This means that the sensor 401 will be ready to use and provide consistent, accurate, and precise results more quickly than conventional designs, which utilize relatively thick layers of membrane 407, and have relatively small areas of the electrode layer in contact with the membrane for material transfer. Further, it should be appreciated that the texture of the membrane surface may be modified using known techniques to improve or optimize wetting properties of the membrane 407.

FIGS. 5a, 5b, and 5c illustrate a two-sided sensor 501 in accordance with the present invention. FIG. 5a illustrates an exemplary top side configuration of an interdigitated electrode structure comprising a working electrode 503 covered by a contoured bio-selective membrane 507 and a reference electrode 504, the working electrode 503 and reference electrode 504 being electrically separated or isolated from one another by a non-conductive passivation layer 509. FIG. 5b illustrates an exemplary bottom side configuration of an interdigitated electrode structure comprising a working electrode 503 covered by a contoured bio-selective membrane 507 and a reference electrode 504, the working electrode 503 and reference electrode 504 being electrically separated or isolated from one another by a non-conductive passivation layer 509.

FIG. 5c illustrates an exemplary cross section of the interdigitated fingers of an exemplary dual-sided sensor structure 501, where the top and bottom sensors are configured to measure different analytes, and are electrically separated or isolated by dual-sided, insulated substrate layer 505. Thus, each side of the dual sided sensor has a unique topography, maximizing the amount of available membrane 507 surface area in a smaller space. It should be appreciated, however, that multiple sensors of the same type may be configured as part of a dual-sided sensor to further increase surface area of the sensor 501 and, thus, lower the detection limit, decrease response time, and potentially provide for a larger linear reaction range. In some implementations, these configurations may increase sensor lifetime by providing redundancy in case of biofouling or damage to components of one or more sensors.

In FIGS. 5a, 5b, and 5c, the overall surface area of the membrane material 507 in contact with the sample is significantly increased relative to the solid-state ion-selective electrode structure shown in FIG. 1 (prior art), thus decreasing sensor response time by least 1%, 2%, 3%, 4%, 5%, 6%, 7%, 8%, 9%, 10%, 15%, 20%, 25%, 30%, 40%, 50% or more. Sensor response time is decreased because transport time across the membrane is decreased as a result of 1) a less thick (or thinner) membrane layer, 2) more surface area of the membrane being in contact with the sample solution, and 3) more surface area of the membrane being in contact with the surface of the electrode. Stated in another way, more of the target substance transported across the membrane makes contact with the working electrode, in comparison to conventional sensor structure illustrated in FIG. 1, in which a relatively small percentage of the target substance transported across the membrane actually makes contact with the surface of the working electrode. Additionally, because the membrane layer is thinner relative to conventional electrode designs, and more of the membrane layer is in contact with the sample, “warm-up” time for the sensor is also reduced by at least 1%, 2%, 3%, 4%, 5%, 6%, 7%, 8%, 9%, 10%, 15%, 20%, 25%, 30%, 40%, 50% or more because more of the target molecule will be transported across the membrane more quickly. This means that the sensor 501 will be ready to use and provide consistent, accurate, and precise results more quickly than conventional designs, which utilize relatively thick layers of membrane, and have relatively small areas of the electrode layer in contact with the membrane for material transfer. Further, it should be appreciated that the texture of the membrane 507 surface may be modified using known techniques to improve or optimize wetting properties of the membrane 507.

It should be appreciated that the dual-sided sensor configuration 501 is illustrated in FIGS. 5a to 5c may be modified so that the top and bottom working electrodes 503 are configured to measure the same analyte, and form a multiplexed sensor array. It should also be appreciated that, in some configurations, portions of the top and bottom sensor may not be electrically isolated from one another. For example, the top and bottom working electrodes may share a common reference electrode that is electrically continuous between the top and bottom surfaces of the substrate.

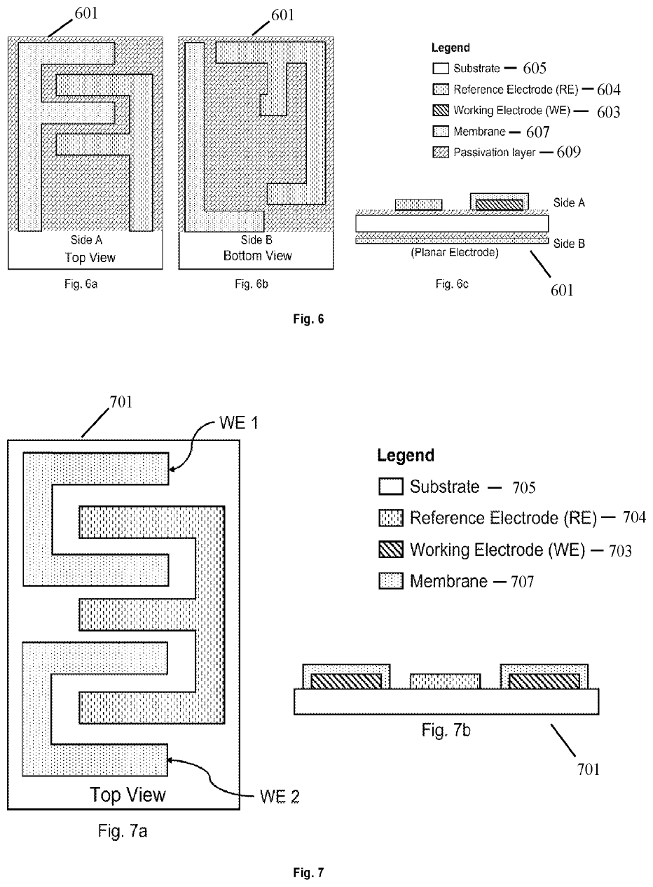

FIGS. 6a, 6b, and 6c illustrate a two-sided sensor 601 in accordance with the present invention. FIG. 6a illustrates an exemplary top side configuration of an interdigitated electrode structure comprising a working electrode 603 covered by a contoured bio-selective membrane 607 and a reference electrode 604. In some embodiments, a passivation layer 609 may electrically isolate the working 603 and reference electrodes 604 from each other and/or the substrate 605. FIG. 6b illustrates an exemplary bottom side configuration of a planar, patterned electrode structure formed on a patterned substrate 605, comprising a working electrode 603 covered by a contoured bio-selective membrane 607 and a reference electrode 604.

FIG. 6c illustrates an exemplary cross section through one of the interdigitated fingers of an exemplary dual-sided sensor structure, where the top and bottom sensors are configured to measure different analytes, and are electrically separated or isolated by dual-sided, insulated substrate layer 605. Thus, each side of the dual sided sensor 601 has a unique topography, maximizing the amount of available membrane 607 surface area for disparate sensors in a smaller space.

In FIGS. 6a, 6b, and 6c, the overall surface area of the membrane material 607 in contact with the sample is significantly increased relative to the solid-state ion-selective electrode structure shown in FIG. 1 (prior art), thus decreasing sensor response time by at least 1%, 2%, 3%, 4%, 5%, 6%, 7%, 8%, 9%, 10%, 15%, 20%, 25%, 30%, 40%, 50% or more. Sensor 601 response time is decreased because transport time across the membrane is decreased as a result of 1) a less thick (or thinner) membrane layer, 2) more surface area of the membrane being in contact with the sample solution, and 3) more surface area of the membrane being in contact with the surface of the electrode. Stated another way, more of the target substance transported across the membrane makes contact with the working electrode, in comparison to conventional sensor structure illustrated in FIG. 1, in which a relatively small percentage of the target substance transported across the membrane actually makes contact with the surface of the working electrode. Additionally, because the membrane layer is thinner relative to conventional electrode designs, and more of the membrane layer is in contact with the sample, “warm-up” time for the sensor is also reduced by at least 1%, 2%, 3%, 4%, 5%, 6%, 7%, 8%, 9%, 10%, 15%, 20%, 25%, 30%, 40%, 50% or more because more of the target molecule will be transported across the membrane more quickly. This means that the sensor will be ready to use and provide consistent, accurate, and precise results more quickly than conventional designs, which utilize relatively thick layers of membrane, and have relatively small areas of the electrode layer in contact with the membrane for material transfer. Further, it should be appreciated that the texture of the membrane surface may be modified using known techniques to improve or optimize wetting properties of the membrane 607.

FIGS. 7a, and 7b illustrate a single-sided sensor 701 having two working electrodes 703, in accordance with the present invention. FIG. 7A illustrates a pair of interdigitated working electrodes 703, each working electrode covered by a contoured bio-selective membrane 707, and a common or shared reference electrode 704. FIG. 7b illustrates an exemplary cross section of the interdigitated fingers of this exemplary single sided, dual working electrode sensor structure, where working electrodes 703 may be configured to sense different analytes or the same analyte, for example, to provide redundancy in case of membrane fouling. Thus, the three-dimensional membrane structure 707 that follows the contours of the working electrode 703 maximizes the amount of available membrane 707 surface area in a smaller space than a conventional interdigitated sensor design. Although the embodiment illustrated in FIGS. 7a and 7b illustrates two working electrodes 703, it should be appreciated that the number of working electrodes may be varied without departing from the scope of the invention.

In FIGS. 7a and 7b, the overall surface area of the membrane material 707 in contact with the sample is significantly increased relative to the solid-state ion-selective electrode structure shown in FIG. 1 (prior art), thus decreasing sensor 701 response time by at least 10%, 20%, 30%, 40%, 50% or more. Sensor 701 response time is decreased because transport time across the membrane is decreased as a result of 1) a less thick (or thinner) membrane layer 707, 2) more surface area of the membrane 707 being in contact with the sample solution, and 3) more surface area of the membrane 707 being in contact with the surface of the electrode. Stated another way, more of the target substance transported across the membrane makes contact with the working electrode, in comparison to conventional sensor structure illustrated in FIG. 1, in which a relatively small percentage of the target substance transported across the membrane actually makes contact with the surface of the working electrode. Additionally, because the membrane layer is thinner relative to conventional electrode designs, and more of the membrane layer is in contact with the sample, “warm-up” time for the sensor is also reduced by at least 1%, 2%, 3%, 4%, 5%, 6%, 7%, 8%, 9%, 10%, 15%, 20%, 25%, 30%, 40%, 50% or more because more of the target molecule will be transported across the membrane more quickly. This means that the sensor 701 will be ready to use and provide consistent, accurate, and precise results more quickly than conventional designs, which utilize relatively thick layers of membrane 707, and have relatively small areas of the electrode layer in contact with the membrane for material transfer. Further, it should be appreciated that the texture of the membrane 707 surface may be modified using known techniques to improve or optimize wetting properties of the membrane 707.

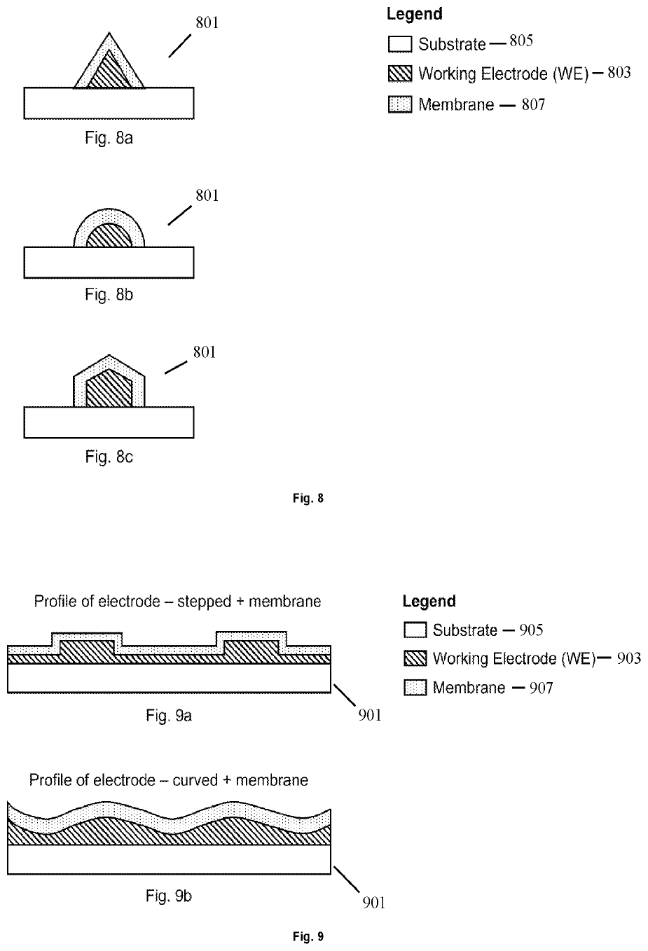

FIGS. 8a, 8b, and 8c illustrate various sensors 801 having working electrodes 803 each having a variety of cross-sectional areas, and membrane 807 topologies or contours that follow the three-dimensional shape/contours of the working electrode 803. Conventionally, working electrodes 803 often have a rectangular or square cross-section. However, modifying the three-dimensional shape of the electrode material 803 that projects from the surface of the substrate may increase surface area, may decrease fouling, and may ease insertion/removal.

FIGS. 9a and 9b illustrate the profile of exemplary interdigitated electrodes 903 of sensors 901 having variable thicknesses of electrode material 903. Varying the depth or thickness of the electrode material 903 that projects from the surface of the substrate 905 may increase surface area of the electrode 903 and/or membrane 907, may decrease fouling, and may ease insertion/removal of the sensor 901.

It should be appreciated that known substrate and electrode patterning techniques may be applied to the sensors described herein in order to provide improved electrical isolation and decrease noise/interference between multiple electrodes without departing from the scope of the invention.

Further, it should be appreciated that aspects of the various embodiments described herein may be combined or applied to any electrode configuration that requires a membrane layer. For example, a potentiometric ion-sensing transducer would require both a working electrode and a reference electrode. The working electrode in a potentiometric ion-sensing transducer is covered by an ion-selective membrane containing an ionophore. However, for transcutaneous implementations of the sensor, biocompatibility of the reference electrode is also required. Thus, the contoured membrane structure of the working electrode may also be applied to the reference electrode using an appropriate biocompatible membrane (not the ion-selective membrane applied to the working electrode) to render, for example, an Ag/AgCl electrode biocompatible. In the event that the either or both of the working and reference electrode materials are already biocompatible, an antifouling membrane may be applied to the surfaces of one or both of the electrodes to create the contoured membrane structure described herein. In an amperometric sensor, for example, any or all of the working electrode, reference electrode, and counter electrode may have the contoured membrane structure disclosed herein without departing from the scope of this invention.

It should be appreciated that the membrane material itself may varied without departing from the scope of the invention, and that a single transducer or sensor may include multiple membrane materials.

Methods of use of the membranes and/or sensors are further described herein and are not particularly limited. For example the methods includes those of: causing and/or creating two-dimensional and/or three-dimensional transport (and/or increasing transport) of an analyte/ion/target of interest through a membrane to a sensor surface: and/or determination an ion in a solution comprising the ion. The methods includes provision of any of the sensors and/or membranes herein described and exposure to a solution containing the target of interest. Electrochemical detection methods of the art can be employed to determine a property of the target such as concentration or determination of the target etc.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments In addition, it is to be understood that the described element(s) may be combined in any suitable manner in the various embodiments.

1. A sensor comprising:
a substrate;
a patterned electrode deposited on the substrate, said patterned electrode having at least one conductive surface; and
a membrane material deposited on the at least one conductive surface of the patterned electrode and not on the planar substrate, wherein said membrane material forms a three-dimensional membrane layer that covers the at least one conductive surface of the patterned electrode.

2. The sensor of claim 1, wherein the patterned electrode is a working electrode.

3. The sensor of claim 1, wherein the membrane material is comprised of an analyte-selective membrane material.

4. The sensor of claim 1, wherein the membrane material is comprised of an ion-selective membrane material.

5. The sensor of claim 1, wherein the substrate is patterned, and the patterned electrode is deposited in the patterned area of the substrate, forming a planar electrode having one conductive surface.

6. The sensor of claim 1, wherein the patterned electrode comprises an interdigitated electrode structure.

7. The sensor of claim 1, wherein the membrane is biocompatible.

8. A sensor, comprising:
a substrate;
a first patterned, three-dimensional working electrode deposited on the substrate, the three-dimensional working electrode having a plurality of surfaces, wherein said plurality of surfaces are not in contact with the substrate;
a reference electrode deposited on the substrate; and
a first membrane layer, wherein said membrane layer is deposited on the plurality of surfaces of the first three-dimensional working electrode.

9. The sensor of claim 8, wherein the membrane layer is not deposited on the reference electrode.

10. The sensor of claim 8, wherein the working electrode and the reference electrode are interdigitated.

11. The sensor of claim 8, further comprising:
a second patterned, three-dimensional working electrode deposited on the substrate, the second three-dimensional working electrode having a second plurality of surfaces, wherein said second plurality of surfaces are not in contact with the substrate; and
a second membrane layer, wherein said second membrane layer is deposited on the plurality of surfaces of the second three-dimensional working electrode.

12. The sensor of claim 8, wherein the first, second, and reference electrodes are interdigitated.

13. The sensor of claim 8, wherein the first membrane layer and the second membrane layer are selective for the same target.

14. The sensor according to claim 8, wherein the first membrane layer and the second membrane layer are selective for different targets.

15. A dual-sided sensor, comprising:
a substrate having a first side and a second side;
a first patterned, three-dimensional working electrode deposited on the first side of the substrate, the three-dimensional working electrode having a plurality of surfaces, wherein said plurality of surfaces are not in contact with the substrate;
a first membrane layer, said first membrane layer being deposited on the plurality of surfaces of the first three-dimensional working electrode;
a second patterned, three-dimensional working electrode deposited on the second side of the substrate, the second three-dimensional working electrode having a second plurality of surfaces, wherein said second plurality of surfaces are not in contact with the substrate;
a second membrane layer, said second membrane layer being deposited on the plurality of surfaces of the second three-dimensional working electrode;
and a reference electrode, said reference electrode not being in contact with the first or second membrane layer.

16. A method of causing two-dimensional or three-dimensional transport (or increasing transport) of an ion through a membrane to a sensor surface, the method comprising the steps of:
providing a sensor as described in claim 8; and
exposing the membrane to a solution comprising the ion.

17. A method of determination an ion in a solution comprising the ion, the method comprising the steps of:
providing a sensor as described in claim 8;
exposing the membrane to the solution comprising the ion; and
electrochemically determining a property of the ion, thereby determining an ion in the solution.

18. The method of claim 17, wherein determined property is the concentration of the ion in the solution.
